(19) United States
(12) Patent Application Publication
(10) Pub. No.: US 2025/0072967 A1
(43) Pub. Date: Mar. 6, 2025

(54) CATHETER SYSTEM WITH MULTIPLE ENERGY SOURCES

(71) Applicant: Shockwave Medical, Inc., Santa Clara, CA (US)

(72) Inventors: Thomas Charles HASENBERG, Campbell, CA (US); Steven Yihlih PENG, Fremont, CA (US); Patrick STEPHENS, Santa Clara, CA (US)

(73) Assignee: Shockwave Medical, Inc., Santa Clara, CA (US)

(21) Appl. No.: 18/817,910

(22) Filed: Aug. 28, 2024

Related U.S. Application Data
(60) Provisional application No. 63/535,302, filed on Aug. 29, 2023.

(51) Int. Cl. A61B 18/26; A61B 17/22; A61B 18/00

(52) U.S. Cl. CPC A61B 18/26; A61B 17/22022; A61B 2018/00577; A61B 2018/263

(57) ABSTRACT

A catheter system for treating stenosis in a body lumen includes a first energy source, a second energy source, and a catheter. The catheter includes an elongate member that is navigable through the body lumen, a first acoustic energy emitter connected to the first energy source and configured to emit acoustic energy when energy is received from the first energy source, and a second acoustic energy emitter connected to the second energy source and configured to emit acoustic energy when energy is received from the second energy source.

### CROSS-REFERENCE TO RELATED APPLICATIONS

This application claims the benefit of U.S. Provisional Patent Application No. 63/535,302, filed Aug. 29, 2023, the entire contents of which is hereby incorporated by reference herein.

### FIELD OF THE DISCLOSURE

The present disclosure relates generally to the field of medical devices and methods, and more specifically to acoustic energy generating assemblies for inclusion in catheter devices used for treating lesions in a body lumen, such as calcified lesions and occlusions in vasculature.

### BACKGROUND

Calcified lesions in body lumens can negatively impact patient health. For example, when calcium builds up in the walls of the coronary arteries, the calcification can restrict blood flow to the heart muscle, which can eventually lead to a heart attack. Catheter devices are one type of device that can be used to treat calcified lesions in a body lumen. When treating lesions with a catheter device, it is important to minimize damage to surrounding soft tissues while still breaking up the lesion as much as possible.

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature.

More recently, the technique and treatment of intravascular lithotripsy (IVL) has been developed, which is an interventional procedure to modify calcified plaque in diseased arteries. The mechanism of plaque modification is through use of a catheter having one or more acoustic shock wave generating sources located within a liquid that can generate acoustic shock waves that modify the calcified plaque. IVL devices vary in design with respect to the energy source used to generate the acoustic shock waves, with two exemplary energy sources being electrohydraulic generation and laser generation.

For electrohydraulic generation of acoustic shock waves, a conductive solution (e.g., saline) may be contained within an enclosure that surrounds electrodes or can be flushed through a tube that surrounds the electrodes. The calcified plaque modification is achieved by creating acoustic shock waves within the catheter by an electrical discharge across the electrodes. This discharge creates one or more rapidly expanding vapor bubbles that generate the acoustic shock waves. These shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding vapor bubble, as well as the acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

The IVL process may be considered different from standard atherectomy procedures in that it cracks calcium but does not liberate the cracked calcium from the tissue. Hence, generally speaking, IVL should not require aspiration nor embolic protection. Further, due to the compliance of a normal blood vessel and non-calcified plaque, the shock waves produced by IVL do not modify the normal vessel tissue or non-calcified plaque. Moreover, IVL does not carry the same degree of risk of perforation, dissection, or other damage to vasculature as atherectomy procedures or angioplasty procedures using cutting or scoring balloons.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with conductive fluid (e.g., using a relatively low pressure of 2-4 atm) so that the balloon expands to contact the lesion but not to a degree that substantively displaces the lesion. Voltage pulses can then be applied across the electrodes of electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can include electrodes disposed within a closed volume other than an angioplasty balloon, such as a cap, balloons of variable compliancy, or other type of enclosure. However, known IVL techniques, while shown to be very effective for treating concentric calcium as well as intimal and subintimal calcium, may not be particularly effective for treating eccentric calcific lesions or chronic total occlusions (CTOs).

Laser atherectomy is an endovascular technique for removing plaque from vessels within the body. Generally, such procedures employ a catheter with forward directed (in a distal direction away from a distal end of the catheter) ultraviolet light energy to break up the buildup of plaque. However, existing laser atherectomy devices have many shortcomings and may not be effective for certain types of lesions, including intimal and subintimal calculi buildup, which is increasingly becoming a greater concern among an aging population.

Further, treating relatively long lesions poses a challenge for known IVL techniques. IVL catheters that are currently available work by first inflating and immobilizing an angioplasty balloon at a lesion and then electrohydraulically generating a series of shock wave pulses. For relatively long lesions, after a first round of shock wave pulses, the angioplasty balloon may be deflated, advanced farther down the lesion, and then reinflated/immobilized for a second round of shock wave pulses. While longer balloons having five or more shock wave generating regions (each region comprising one or more electrode pairs) are available, building out even longer balloons and electrode assemblies presents challenges in manufacturing that make such devices impractical.

Therefore, there is a need for improved IVL devices to treat cardiovascular lesions.

### SUMMARY

According to various embodiments, a catheter system includes a catheter having multiple acoustic energy emitters and one or more energy sources connected to the acoustic energy emitters. According to various embodiments, a catheter system includes acoustic energy emitters that emit acoustic energy having different sonic properties for targeting different kinds of lesions and/or tissue. The catheters and catheter systems described herein may provide versatility in treating stenotic lesions of complex morphologies such as those including multiple tissue types or lesions in very long and/or narrow body lumens.

According to various embodiments, a catheter for treating stenosis in a body lumen includes: an elongate member; an enclosure sealed to a distal region of the elongate member and fillable with a fluid; a forward-firing acoustic energy emitter located on the elongate member at least partially outside of the enclosure on a distal end of the enclosure; and a radially-firing acoustic energy emitter located on the elongate member at least partially inside of the enclosure.

The forward-firing acoustic energy emitter may be optically connected to a light energy source. For example, the forward-firing acoustic energy emitter may be optically connected by an optical fiber to the light energy source.

The light energy source may include a laser, and the forward-firing emitter may include a distal end of an optical fiber.

The light energy source may emit infrared light. In some embodiments, the light energy source may emit near infrared light.

The radially-firing acoustic energy emitter may be electrically connected to a voltage pulse generator.

The radially-firing acoustic energy emitter may include an electrode pair.

A first electrode of the electrode pair may include a conductive surface of a band and a second electrode of the electrode pair may include a conductive portion of an elongate conductive member.

In some examples, the radially-firing acoustic energy emitter includes more than one electrode pair.

The forward-firing acoustic energy emitter may be electrically connected to a voltage pulse generator.

The forward-firing acoustic energy emitter may include an electrode pair.

The forward-firing acoustic energy emitter may include both an electrode pair electrically connected to a voltage pulse generator and a light emitting region of an optical fiber that is optically connected to a light energy source.

The radially-firing acoustic energy emitter may be optically connected to a light energy source.

The light energy source may emit an infrared laser light.

The light energy source may emit a near infrared laser light.

The light energy source may emit an ultraviolet light. In some examples, the fluid may include a contrast agent having high absorbance of the ultraviolet light

The catheter may include a first radially-firing acoustic energy emitter and a second radially-firing acoustic energy emitter.

The first radially-firing acoustic energy emitter may be electrically connected to a voltage pulse generator and the second radially-firing acoustic energy emitter may be optically connected a light energy source.

The forward-firing acoustic energy emitter and the radially-firing acoustic energy emitter may be connected to different types of energy sources.

The forward-firing acoustic energy emitter and the radially-firing acoustic energy emitter may be connected to a single energy source.

The forward-firing acoustic energy emitter and the radially-firing acoustic energy emitter may be connected to a single energy source and be electrically connected in series.

The forward-firing acoustic energy emitter and the radially-firing acoustic energy emitter may be connected to separate channels of a high-voltage generator.

According to aspects of the disclosure, a catheter for treating stenosis in a body lumen includes: an elongate member; an enclosure sealed to a distal region of the elongate member; a longitudinally movable member mounted at least partially around the elongate member and located inside of the enclosure; a forward-firing acoustic energy emitter located on the elongate member at least partially outside of the enclosure on a distal side of the enclosure; and a radially-firing acoustic energy emitter located on the longitudinally movable member at least partially inside of the enclosure.

The forward-firing acoustic energy emitter may include a distal end of an optical fiber that extends from a laser source.

The radially-firing acoustic energy emitter may include a distal end of an optical fiber that extends from a laser source.

The laser source may generate a laser having a wavelength suitable for treating calcified lesions.

The laser source may generate a laser having a wavelength suitable for treating tissue softer than calcium.

The laser source may generate a laser having a wavelength suitable for absorption by target tissue.

The laser source may generate a laser having a wavelength suitable for absorption by water.

According to aspects of the disclosure, a catheter for treating stenosis in a body lumen includes: an elongate member; an enclosure sealed to a distal region of the elongate member and having a length no less than 30 mm; and an emitter assembly that is movable along a longitudinal direction of the enclosure, the emitter assembly being at least partially located within the enclosure and including one or more light output regions.

According to aspects of the disclosure, a method of treating a lesion in a vessel includes: advancing a catheter into the vessel until a distal end of the catheter is positioned proximate the lesion; delivering energy to a distal acoustic energy emitter of the catheter; advancing the catheter such that an enclosed radially-firing acoustic energy emitter of the catheter is adjacent the lesion; delivering energy to the enclosed radially-firing acoustic energy emitter to generate acoustic energy to further treat the lesion; and generating one or more pressure waves from the enclosed radially-firing acoustic energy emitter.

The radially-firing acoustic energy emitter may include one of an electrode pair and a distal end of an optical fiber.

The method may further include, before the step of delivering energy to the enclosed radially-firing acoustic energy emitter, inflating the enclosure of the catheter; after the step of delivering energy to the enclosed radially-firing acoustic energy emitter, moving the enclosed radially-firing acoustic energy emitter in a longitudinal direction of the catheter; and repeating the step of generating one or more pressure waves from the radially-firing acoustic energy emitter.

The acoustic energy may be a shock wave.

The lesion may be a chronic total occlusion.

The method may include the step of imaging the lesion. Imaging may be accomplished, for example, by fluoroscopy, intravascular ultrasound, or optical coherence tomography.

The method may further include the step of tuning acoustic energy properties of the distally-firing acoustic energy emitter. For example, the sonic output of the distally-firing acoustic energy emitter may be adjusted. The acoustic energy properties may be tuned to match physical properties of a target tissue.

According to aspects of the disclosure, a catheter system for treating stenosis in a body lumen includes: a first energy source; a second energy source; and a catheter including an elongate member configured to be navigated through the body lumen, a first acoustic energy emitter connected to the first energy source and configured to emit acoustic energy when energy is received from the first energy source, and a second acoustic energy emitter connected to the second energy source and configured to emit acoustic energy when energy is received from the second energy source.

The catheter may include an enclosure and the first acoustic energy emitter and the second acoustic energy emitter may be enclosed in the enclosure.

The first energy source may be a voltage pulse generator and the second energy source is a laser light source.

The first energy source may be a first voltage pulse generator and the second energy source may be a second voltage pulse generator configured to generate voltage pulses having different electrical properties than voltage pulses generated by the first voltage pulse generator.

The first acoustic energy emitter and the second energy emitter may both be unenclosed.

The first energy source may be a first laser light source and the second energy source may be a second laser light source that generates light having different light energy properties than the light generated by the first laser light source.

The catheter may further include a third energy source, wherein the catheter includes a third acoustic energy emitter connected to the third energy source.

### DESCRIPTION OF THE FIGURES

Illustrative aspects of the present disclosure are described in detail below with reference to the following figures. It is intended that the embodiments and figures disclosed herein are to be considered illustrative and exemplary rather than restrictive.

FIG. 1 illustrates a catheter system for treating a stenotic lesion in a body lumen, according to aspects of the disclosure.

FIG. 2A illustrates a distal region of a catheter connected to more than one energy source, according to aspects of the disclosure.

FIG. 2B illustrates a cross-sectional view of the catheter of FIG. 2A.

FIG. 2C illustrates a magnified cross-sectional view of an emitter of the catheter of FIGS. 2A and 2B.

FIG. 3A illustrates a distal region of a catheter including enclosed and unenclosed light-based acoustic energy emitters, according to aspects of the disclosure.

FIG. 3B illustrates a cross-sectional view of the catheter of claim 3A.

FIGS. 4A and 4B illustrate a catheter having longitudinally translatable enclosed emitters, according to aspects of the disclosure.

FIGS. 4C and 4D illustrate another catheter having longitudinally translatable enclosed emitters, according to aspects of the disclosure.

FIG. 5 illustrates a cross-sectional view of a catheter including optical fibers as energy guides, according to aspects of the disclosure.

FIG. 6. illustrates an enclosed electrohydraulic emitter, according to aspects of the disclosure.

FIG. 7 illustrates an unexposed electrohydraulic emitter, according to aspects of the disclosure.

FIG. 8 illustrates a computing system for use with a catheter system, according to aspects of the disclosure.

FIG. 9 illustrates a method of treating a stenotic lesion, according to aspects of the disclosure.

FIG. 10 illustrates another method of treating a stenotic lesion, according to aspects of the disclosure.

### DETAILED DESCRIPTION

The following description is presented to enable a person of ordinary skill in the art to make and use the various embodiments and aspects thereof disclosed herein. Descriptions of specific catheters, systems, methods, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement. Furthermore, numerical designators such as “first,” “second,” “third,” “fourth,” etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). In some contexts, one or more electrode pairs may also be referred to as an electrode assembly. In the context of the present disclosure, the term “emitter” broadly refers to the region of an electrode assembly where the current transmits across the electrode pair, generating a shock wave. The term “emitter sheath” or “emitter band” (which are used interchangeably) refers to a sheath/band of conductive material that may form one or more electrodes of one or more electrode pairs, thereby forming a location of one or more emitters.

Components of emitters, including electrodes and emitter sheaths/bands, may be formed from a metal, such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or a mixture of such materials.

In some embodiments, an IVL catheter is a so-called “rapid exchange-type” (“Rx”) catheter provided with an opening portion through which a guide wire is guided (e.g., through a middle portion of a central tube in a longitudinal direction). In other embodiments, an IVL catheter may be an “over-the-wire-type” (“OTW”) catheter in which a guide wire lumen is formed throughout the overall length of the catheter, and a guide wire is guided through the proximal end of a hub.

As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs can be found in U.S. Pat. Nos. 8,888,788, 9,433,428, and 10,709,462, in U.S. Publication No. 2021/0085383, and in U.S. patent application Ser. No. 18/586,299, all of which are incorporated herein by reference in their entireties. Other catheter designs have improved the delivery of shock waves, for instance, by specific electrode construction and configuration thereby directing shock waves in a forward direction to break up tighter and harder-to-cross occlusions in vasculature. Examples of forward-biased or firing-firing catheter designs can be found in U.S. Pat. Nos. 10,966,737, 11,478,261, and 11,596,423, in U.S. Publication Nos. 2023/0107690 and 2023/0165598, and in U.S. patent application Ser. Nos. 18/524,575 and 18/680,853, all of which are incorporated herein by reference in their entireties.

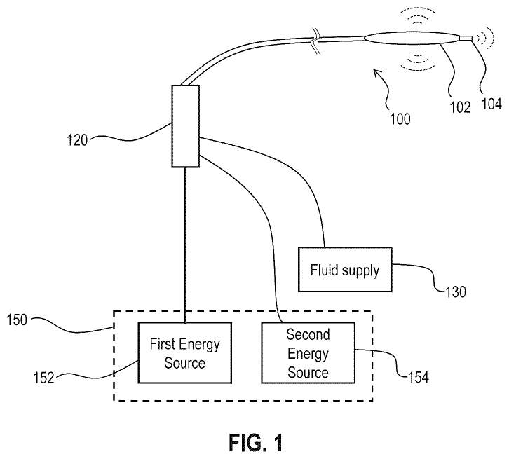

FIG. 1 illustrates a shock wave catheter system 10 with multiple energy sources, according to aspects of the disclosure. System 10 includes a shock wave catheter 100, which is connected to a first energy source 152 and a second energy source 154. Shock wave catheter 100 includes an enclosure 102, and acoustic energy emitters may be located inside and/or outside of enclosure 102. For example, one or more acoustic energy emitters may be located proximate a distal end 104 of the catheter. Catheter 100 is configured to emit acoustic energy generated from the delivery of energy from one or both of first and second energy sources 152, 154 to emitters inside or outside of enclosure 102. Accordingly, acoustic energy may be emitted distally (which may be referred to herein as “forward-firing”) and/or radially (which may be referred to herein as “radially-firing” or “side-firing”). Shock wave catheter system 10 may include a fluid supply 130 connected via a hub 120 to catheter 100. Fluid supply 130 may supply conductive fluid (e.g., saline, contrast media, or a mixture of saline and contrast media).

Acoustic energy emitters may emit shock waves. In some embodiments, acoustic energy emitters generate cavitation bubbles whose collapse generates acoustic pressure waves and/or microjets.

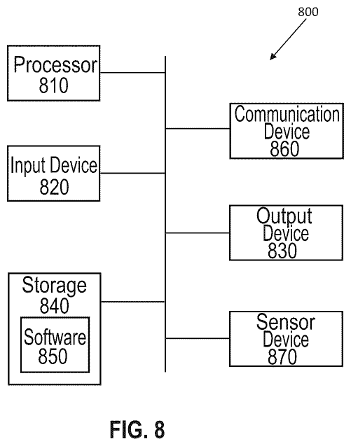

First and second energy sources 152, 154 may include a high voltage pulse generator, a laser light source, or another energy source. Acoustic energy (e.g., shock waves and/or cavitation bubbles) may be generated from an electrohydraulic emitter (e.g., one or more electrode pairs that generate acoustic energy), light energy emitter, piezo-electric emitter, or an electromagnetic emitter (e.g., an acoustic energy generator that uses electromagnetic forces to move a fluid to create shock waves and/or cavitation bubbles). In some embodiments, first energy source 152 is connected to one or more unenclosed emitters (those at or proximate distal end 104) of catheter 100, and second energy source 154 is connected to one or more emitters enclosed within enclosure 102. For example, if one of the energy sources is a high voltage pulse generator, the energy source may be connected electrically via conductive members (e.g., conductive wires) to electrode pairs of the acoustic energy emitters. If one of the energy sources is a laser light source, the energy source may be optically connected by light transmitting members (e.g., optical fibers) to the acoustic energy emitters, which may comprise light emitting regions of the light transmitting members. First energy source 152 and second energy source 154 may be part of an energy source subsystem 150 including various processors and software as further described below with respect to FIG. 8.

In some embodiments, an acoustic energy catheter system may include more than two energy sources (e.g., three, four, five, or more energy sources). Such a system may be suitable for treating larger body lumens as including additional elongate energy guides may substantially increase the catheter's crossing profile. In some embodiments, an acoustic energy catheter system including a catheter with forward-firing and radially-firing emitters has a single energy source that provides energy to both unenclosed (e.g., forward-firing) and enclosed (e.g., radially-firing) emitters.

In the case of energy supplied by a high voltage pulse generator, a voltage pulse applied by the energy source may generate, by an electrohydraulic mechanism, one or more shock waves at the emitter. A voltage pulse from a high voltage pulse generator, which may also be referred to herein as voltage sources or voltage pulse generators, is typically in the range of from about five hundred to three thousand volts (500 V-3,000 V). In some implementations, the voltage pulse applied by the voltage source can be up to about ten thousand volts (10,000 V) or higher than ten thousand volts (10,000 V). The pulse width of the applied voltage pulses ranges between two microseconds and six microseconds (2-6 μs). The repetition rate or frequency of the applied voltage pulses may be between about 1 Hz and 10 Hz. The total number of pulses applied by the energy source may be, for example, sixty (60) pulses, eighty (80) pulses, one hundred twenty (120) pulses, three hundred (300) pulses, or up to five hundred (500) pulses, or any increments of pulses within this range. Alternatively, or additionally, in some examples, the energy source may be configured to deliver a packet of micro-pulses having a sub-frequency between about one hundred hertz to ten kilohertz (100 Hz-10 kHz). The preferred voltage, repetition rate, and number of pulses may vary depending on, e.g., the size of the lesion, the extent of calcification, the size of the blood vessel, the attributes of the patient, or the stage of treatment. For instance, a physician may start with low energy shock waves and increase the energy as needed during the procedure, or vice versa. The magnitude of the shock waves can be controlled by controlling the voltage, current, duration, and repetition rate of the pulsed voltage from the energy source.

For embodiments using light energy, the energy source, in one or more embodiments, is selected from a laser having a wavelength of 100 nm to 10,000 nm. For generating acoustic pressures from light energy, certain light energy sources may be favored. For example, a neodymium-doped yttrium aluminum garnet (Nd:YAG) laser, a thulium:yttrium-aluminum-garnet (Tm:YAG) laser, a holmium:yttrium-aluminum-garnet (Ho:YAG) laser, or an erbium:yttrium-aluminum-garnet (Er:YAG) laser may be used, although others are possible. For example, a Er:YAG may be selected for its high water absorption.

In one or more embodiments, the catheter system 10 having both forward-firing and radially-firing emitters includes an ultraviolet laser as the energy source for the forward-firing emitter. In some embodiments, system 10 includes an excimer laser as the energy source for the one or more forward-firing emitters. Such lasers may have a wavelength of 126 nm to 351 nm. In some embodiments, the wavelength is about 308 nm. The wavelength may be selected based on the type of tissue to be ablated.

In some embodiments, the laser wavelength is tunable to be optimized for a particular tissue type. In some embodiments, one or both of the energy sources 152, 154 include a wavelength switchable light energy source. The wavelength switchable light energy source may, for example, include multiple light energy sources emitting different wavelengths of light. Depending on the type (e.g., fibrotic, thrombic, or calcific), size, or the makeup of the fluid (e.g., blood, saline, contrast solution), an optimal wavelength of light from the multiple light energy sources may be selected for a procedure. In some embodiments, a first light energy source emitting light having a first wavelength may be selected if tissue modification by mainly acoustic effects is desired and a second light energy source emitting light having a second wavelength may be selected if tissue modification by direct tissue ablation from absorption of light by tissue is desired. Once selected, the light energy source emitting the selected wavelength of light may be optically coupled to a light energy guide (e.g., an optical fiber or an optical fiber bundle) to deliver the light energy to one or more emitters.

For soft tissue modification by direct tissue ablation, water may be used as a chromophore for light absorption. In some examples, light having a wavelength of 1400 nm-1520 nm or 1900 nm-2100 nm is used to modify soft tissue (to target water absorption peaks at 1470 nm or 2000 nm). For vascular tissue, oxy-hemoglobin may be used as a chromophore for light absorption. In some examples, light having a wavelength of 410 nm-470 nm or 500 nm-560 nm is used to modify vascular tissue. In some examples, light having a wavelength of 480 nm-500 nm, 610 nm-650 nm, or 800 nm-900 nm is used to target calcified tissue. In some examples, light having a wavelength around 405 nm-1064 nm, 1470 nm, 1950 nm, 2000 nm, 2020 nm, 2120 nm, 2940 nm, or 9300 nm-10,600 nm is used to modify soft tissue (e.g., tissue including protein and water). In some examples, light having a wavelength of around 2940 nm or 9000 nm-10000 nm is used to modify denser tissue (e.g., tissue including calcium minerals).

In some embodiments, one or both of the energy sources 152, 154 is an infrared or visible laser light source, for example a Tm:YAG laser, InGaAs diode laser, a Nd:YAG laser, a pulsed dye laser, a holmium YAG laser, or a thulium fiber laser, or any other suitable laser. The light transmitted into the optical fibers of the catheter from the one or both light energy source may have a near-infrared wavelength, that is, a wavelength between about 760 nm and about 1500 nm. For example, the light transmitted into the optical fibers of catheter 102 from light energy source 106 may have a wavelength of about 800 nm, 820 nm, 840 nm, 860 nm, 880 nm, 900 nm, 920 nm, 940 nm, 960 nm, 980 nm, 1000 nm, 1020 nm, 1040 nm, 1060 nm, 1080 nm, 1100 nm, 1120 nm, 1140 nm, 1160 nm, 1180 nm, 1200 nm, 1220 nm, 1240 nm, 1260 nm, 1280 nm, 1300 nm, 1320 nm, 1340 nm, 1360 nm, 1380 nm, 1400 nm, 1420 nm, 1440 nm, 1460 nm, 1480 nm, or 2200 nm. In some embodiments, one or both of the energy sources 152, 154 is a laser light source that transmits light having a wavelength of about 1064 nm. In some embodiments, one or both of the energy sources 152, 154 is a laser light source that transmits light having a wavelength of about 1470 nm. Advantageously, near infrared light exhibits strong absorption in water so may be suitable for use with aqueous solutions such as saline for generating acoustic energy. But other wavelengths of light may be chosen; for example, an ultraviolet light energy source, such as those described above, may be selected in combination with a contrast media.

A light energy source may be configured to provide pulses of light. For enclosed emitters, the width of each light pulse may be less than the time required for a bubble to reach equilibrium in the fluid contained in an enclosure if the fluid was free (e.g., not enclosed). In some embodiments, the width of each pulse is between 1 ns and 30 ns, between 5 ns and 25 ns, between 10 ns and 25 ns, or between 15 ns and 20 ns, for example about 16 ns, about 17 ns, about 18 ns, about 19 ns, or about 20 ns. In other embodiments, the width of each pulse is between 50 ns and 500 μs, for example 100 ns, 500 ns, 1 μs, 50 μs, 75 μs, 100 μs, 150 μs, 200 μs, 250 μs, 300 μs, 350 μs, 400 μs, or 450 μs. The peak power of each light pulse can be between 100 W and 500 W, for example approximately 150 W, 200 W, 250 W, 300 W, 350 W, 400 W, or 450 W. The pulse repetition rate may be between about 100 Hz and 1 kHz, for example about 150 Hz, 200 Hz, 250 Hz, 300 Hz, 350 Hz, 400 Hz, 450 Hz, 500 Hz, 550 Hz, 600 Hz, 650 Hz, 700 Hz, 710 Hz, 720 Hz, 730 Hz, 740 Hz, 750 Hz, 760 Hz, 770 Hz, 780 Hz, 790 Hz, 800 Hz, 810 Hz, 820 Hz, 830 Hz, 840 Hz, 850 Hz, 900 Hz, or 950 Hz. In comparison to electrohydraulic acoustic energy emitters described above, acoustic energy emitters powered by a light energy source may be capable of emitting a substantially higher number of acoustic energy pulses before degradation of the emitter and device. In some embodiments, the light energy source may provide up to one hundred thousand (100,000) pulses of light to the device. In some embodiments, the light energy source may provide more than one hundred thousand (100,000) pulses of light to the device.

In some embodiments, catheter 100 may include a plurality of forward firing and/or radially firing emitters that are optically connected to two or more light energy sources. The two or more light energy sources may generate different wavelengths of light. In some embodiments, a first light energy source may generate light pulses suitable for ablating tissue, and a second light energy source may generate light pulses suitable for generating acoustic energy. For instance, the first light energy source may emit light having a wavelength absorbed by a target tissue, and the second light source may emit light having a different wavelength that is absorbed by media (e.g., saline, plasma) surrounding the emitter(s).

In some embodiments, an IVL catheter is a so-called “rapid exchange-type” (“Rx”) catheter provided with an opening portion through which a guide wire is guided (e.g., through a middle portion of a central tube in a longitudinal direction). In other embodiments, an IVL catheter may be an “over-the-wire-type” (“OTW”) catheter in which a guide wire lumen is formed throughout the overall length of the catheter, and a guide wire is guided through the proximal end of a hub.

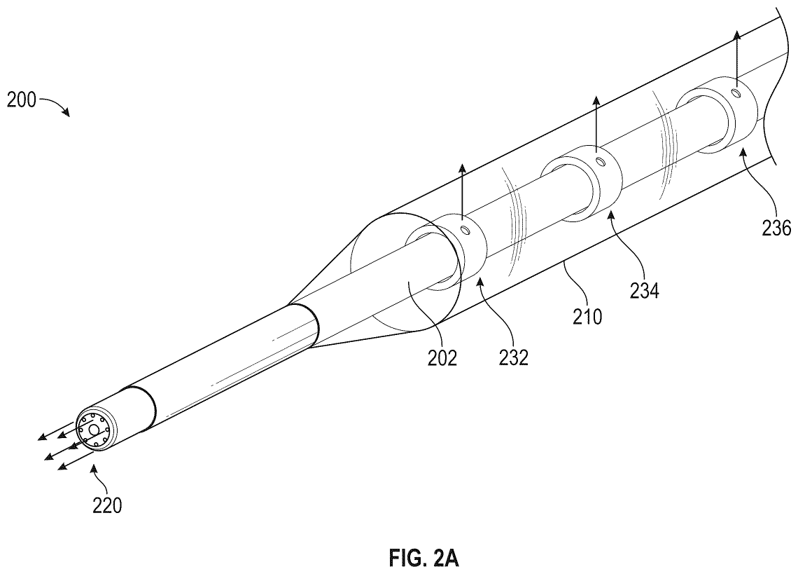

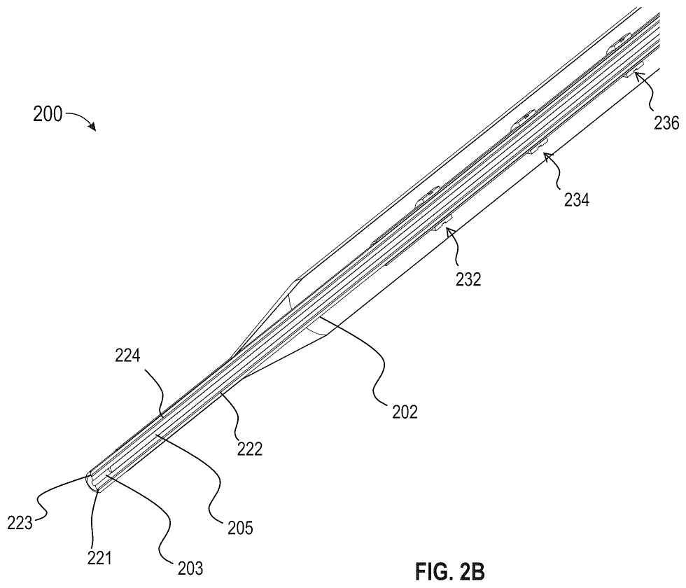

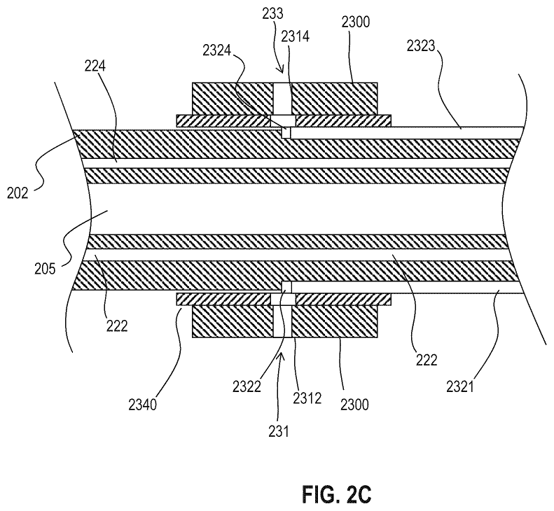

FIG. 2A illustrates a perspective view, FIG. 2B illustrates a longitudinal cross-sectional view, and FIG. 2C illustrates a magnified longitudinal cross-sectional view of a distal end of a catheter 200, according to aspects of the disclosure. Catheter 200, which may be included as part of a system such as acoustic energy emitting catheter system 10 shown in FIG. 1, includes one or more acoustic energy emitters 220 located on a distal end of, and at least partially outside of an enclosure 210. Acoustic energy emitters 220 may emit forward-directed acoustic energy generated by laser light energy (e.g., the near infrared or infrared wavelength light energy described above) at the distal end of catheter 200 for opening of relatively tighter (e.g., more occluded) calcified lesions. In some embodiments, acoustic energy emitters 220 generate one or more distally propagating cavitation bubbles whose collapse emit acoustic energy (e.g., from shock waves and/or microjets) that may disrupt a lesion located distally of enclosure 210.

Optical fibers 222, 224 that deliver laser light energy from a laser light source are located inside or adjacent to an elongate shaft 202 of catheter 200. Each optical fiber 222, 224 can be a flexible optical fiber or a fiber bundle including a plurality of flexible optical fibers. Additional optical fibers may be included in catheter 200 and circumferentially distributed about a central guide wire lumen 203. Optical fibers 222, 224 may extend distally from a laser light source to distal emitters 220 including optical fiber ends 221, 223 for emitting light energy distally. In such embodiments, optical fibers ends 221, 223 may emit light energy for generating acoustic waves upon absorption of the light energy by surrounding media and/or for ablating a target tissue.

As shown in FIGS. 2A, 2B, and 2C, catheter 200 includes a plurality of acoustic energy emitters 232, 234, 236 (e.g., shock wave generating regions) mounted on a fixed or longitudinally movable elongate shaft 202 and enclosed within enclosure 210 to target calcified lesions of a body lumen. Enclosure 210 (e.g., a balloon) may range from 2 mm to 5 mm in diameter when inflated; enclosure 110 may be longer than those used with existing commercially available IVL devices. In some embodiments, the enclosure may include an angioplasty balloon having a working length greater than 80 mm. In some embodiments, the enclosure may include an angioplasty balloon having a working length greater than 10 cm in length. In one or more embodiments, the working length of the enclosure (e.g., the generally tubular region of an angioplasty balloon) can be as long as 300 mm. In some embodiments, the working length of the enclosure is up to 200 mm. In one or more embodiments, the working length is no less than 30 mm. In other embodiments, the working length is no less than 50 mm.

As shown in the magnified cross-sectional view of FIG. 2C, an electrohydraulic acoustic energy emitter located inside of enclosure 210, such as emitters 232, 234, 236, may include one or more electrode pairs 231, 233. First electrodes of the electrode pairs 231, 233 may include outer electrodes formed by conductive surfaces 2312, 2314 of a band 2300. As shown in FIG. 2C, conductive surfaces 2312, 2314 may be located on apertures of band 2300. In other embodiments, conductive surfaces may be located on other regions, such as a distal edge or a proximal edge of band 2300. Additional apertures may be provided in band 2300 to form additional conductive surfaces and electrode pairs.

Second electrodes of electrode pairs 231, 233 may include conductive regions 2322, 2324 of elongate conductive members 2321, 2323 and may be provided as inner electrodes of electrode pairs 231, 233. Conductive regions 2322, 2324, in some examples, include exposed (uninsulated) regions of elongate conductive members 2321, 2323. In other examples, one or both inner electrodes of electrode pairs 231, 233 may be formed by other conductive members (such as a stainless steel sheath) that is directly and electrically connected to elongate conductive members 2321, 2323. Elongate conductive members 2321, 2323, in turn, may be electrically connected to positive and negative terminals of a high voltage pulse generator. In various embodiments, elongate conductive members 2321, 2323 are insulated conductive wires.

In electrohydraulic examples of acoustic wave emitters, each electrode pair includes a gap separating the two electrodes. When a high voltage pulse (as high as 10 kV) is delivered to elongate conductive members 2321, 2323, current may flow (a) from a first inner electrode (e.g., conductive region 2322), (b) across a gap of a first electrode pair (e.g., electrode pair 231), (c) to a first outer electrode (e.g., conductive surface 2312 of band 2300), (d) along band 2300 to a second outer electrode (e.g., conductive surface 2314 of band 2300), and (e) across a gap of a second electrode pair (e.g., conductive region 2324). An insulative sleeve 2340 may be positioned between elongate shaft 202 and band 2300. Insulative sleeve 2340 may be provided with apertures aligned with apertures of band 2300 and formed from an insulating polymer such as polyimide or polyurethane. At each electrode pair 231, 233, a shock wave may be generated when current is delivered across the corresponding gap. Accordingly, each acoustic energy emitter 232, 234, 236 may include more than one electrode pairs connected in series to each other. Further, two or more acoustic energy emitters 233, 234, 236 may be connected in series to each other such that a single high voltage pulse generates a shock wave at each electrically connected electrode pair and emitter.

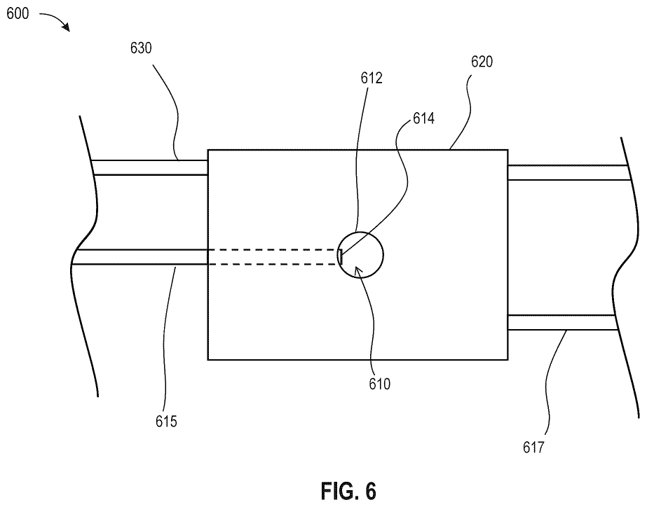

FIG. 6 illustrates an emitter 600 that may form an enclosed emitter, such as emitters 232, 234, 236, where emitter 600 is electrically connected in series to one or more emitters. Emitter assembly 600 includes an electrode pair 610. Electrode pair 610 may include a first electrode 612 and a second electrode 614. First electrode 612 may be formed by a surface of a conductive outer member 620. In some embodiments, the conductive outer member may be a conductive band having an aperture whose surface forms first electrode 612. Second electrode 614 may be formed by a conductive inner member 615, which is electrically connected to a high voltage generator. Wire 617 may electrically connect conductive outer member 620 to another adjacent conductive outer member. For example, wire 617 may form an inner electrode at the adjacent conductive member. Wire 630 may be insulated from and extend under conductive outer member 620 to serve as a return wire from the adjacent conductive outer member to the high voltage generator.

Other electrode pair configurations for enclosed emitters are possible. In some examples, electrode pairs may be formed by uninsulated portions of conductive wires separated by a gap, where the conductive wires are connected to positive and negative terminals of a high voltage generator. Electrode pairs, in other examples, may be formed by adjacent conductive edges of spaced apart bands or rings.

FIGS. 2A, 2B, and 2C depict the plurality of enclosed acoustic energy emitters 232, 234, 236 as electrohydraulic shock wave emitters, but, in other embodiments, and as described in more detail below, these acoustic energy generating regions may generate shock waves (or pressure pulses) from a light energy source (e.g., a laser) from the same or a different light energy source as the one optically connected to shock wave emitters 220.

Elongate shaft 202 includes a guidewire lumen 203 for a guidewire 205 by which the catheter can be delivered to the treatment site. In one or more embodiments, catheter 200 includes additional lumina for delivering fluid (e.g., conductive fluid) to fill or inflate the enclosure. In one or more embodiments, catheter 200 includes additional lumina that extend proximate distal emitters 220 to infuse fluid to the distal emitters 220 and/or aspirate the treatment site during or after treatment.

Components of electrohydraulic emitters, including electrodes and emitter sheaths/bands, may be formed from a metal, such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or a mixture of such materials.

In other embodiments, the emitter types may be flipped such that an electrohydraulic emitter fires forward-directed cavitation bubbles (e.g., using electrode designs described in U.S. Pub. No. 2023/0165598, and radial pressure waves (e.g., shock waves) are generated by a laser in the enclosure. Other combinations are possible (such as both forward and radial firing electrohydraulic emitters or both forward and radial firing laser emitters). Other types of pressure wave generating emitters are included in other embodiments, such as those employing piezoelectric elements.

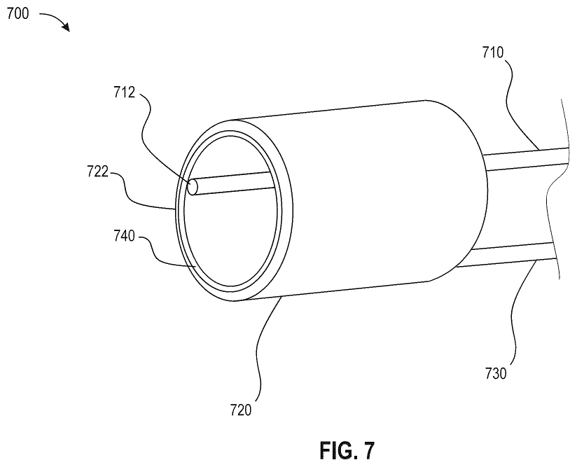

In some embodiments, an acoustic energy catheter includes distal unenclosed electrohydraulic emitters. Such catheters may include emitter 700, illustrated in FIG. 7, as a distal emitter. Emitter 700 includes a conductive region 712 of conductive elongate member 710 as a first electrode of an electrode pair. Elongate member 710 may be electrically connected to a high voltage generator. A conductive edge 722 of band 720 may form a second electrode of the electrode pair. A return wire 740 may electrically connect band 720 to the high voltage generator. An insulating layer 740 may ensure that the electrodes are not in contact with each other. Although FIG. 7 illustrates the conductive region 712 as an inner electrode and conductive edge 722 as an outer electrode, other configurations are possible. In some embodiments, a conductive region of the elongate member 710 may form the outer electrode and the band 720 may form an inner electrode. In some embodiments, the band 720 may include a slit or slot and the elongate member 710 may be positioned within the slit or slot. Further, distal emitter 700 may include more than one electrode pair. In some embodiments, a distal emitter may include two electrode pairs connected in series.

In some embodiments, wires 710 and 730 extend along or through dedicated lumina of an elongate shaft (e.g., shaft 202 described above) to a high voltage generator. A catheter having distal unenclosed electrohydraulic emitters and enclosed electrohydraulic emitters may be used with a high voltage generator having separate dedicated channels for the unenclosed and enclosed emitters. In some embodiments, unenclosed emitters may be connected to a first high voltage generator and enclosed emitters may be connected to a second high voltage generator. Such systems may allow separate control of unenclosed and enclosed emitters and may be necessary to accommodate the different power requirements or safety requirements of these methods of action. In some embodiments, unenclosed emitters are provided with lower amplitude voltage pulses than enclosed emitters. In some embodiments, unenclosed emitters are provided with a different frequency of pulsing than enclosed emitters. In some embodiments, unenclosed emitters are provided with a higher frequency and lower amplitude pulses than enclosed emitters.

In some embodiments, unenclosed distal emitter 700 is electrically connected to one or more enclosed emitters by wires 710, 730. In such embodiments, a high-voltage pulse may generate acoustic energy (e.g., from shock waves or cavitation bubble collapse) at electrode pairs of unenclosed distal emitter 700 and enclosed emitters. Unenclosed distal emitter 700 may be electrically connected in series to one or more enclosed emitters by wires 710, 730.

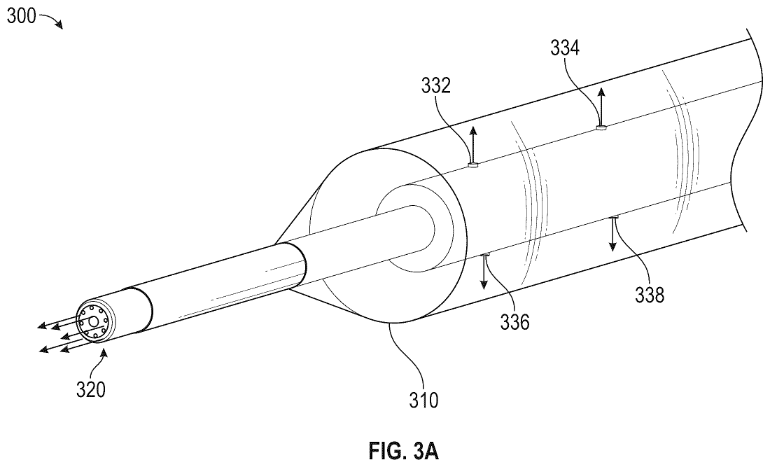

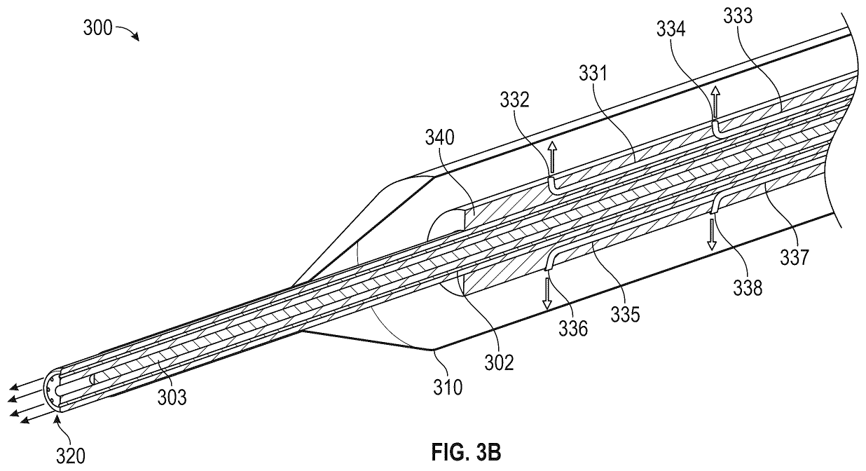

FIG. 3A illustrates a perspective view and FIG. 3B illustrates a perspective cross-sectional view of an acoustic energy emitting catheter 300, according to aspects of the disclosure. Catheter 300, similar to catheter 200, includes one or more unenclosed acoustic energy emitters 320 and enclosed acoustic energy emitters 332, 334, 336, 338 enclosed in fluid-filled enclosure 310. In contrast to catheter 200, each of the enclosed acoustic energy emitters of catheter 300 are formed by one or more light emitting regions of an optical fiber. For example, emitter 332 includes a distal end of an optical fiber 331; emitter 334 includes a distal end of an optical fiber 333; emitter 336 includes a distal end of an optical fiber 335; and emitter 338 includes a distal end of an optical fiber 337. In other embodiments, one or more of the enclosed emitters may be formed by an evanescent portion of an optical fiber that is not necessarily a distal end of the optical fiber (e.g., a portion of the optical fiber having thinned cladding regions). In some embodiments, each emitter may include light emitting regions of a plurality of optical fibers (e.g., distal ends of an optical fiber bundle).

Optical fibers 331, 333, 335, 337 may extend substantially longitudinally along the length of catheter 300, parallel to elongate shaft 302. Each optical fiber 331, 333, 335, 337 may have, proximate its distal terminus, a curved region such that a distal end of each optical fiber 331, 333, 335, 337 is angled away from a distal end of catheter 300. In some embodiments, each optical fiber 331, 333, 335, 337 includes, proximate its distal terminus, a curved region such that a distal end of each optical fiber 331, 333, 335, 337 is angled away from a distal end of catheter 300 by an angle of 5 degrees to 90 degrees. In some embodiments, this angle is less than 90 degrees. In some embodiments, the distal end of one or more of optical fibers 331, 333, 335, 337 are positioned such that light is emitted from the distal end in a transverse direction (radially outward) from elongate shaft 302. In some embodiments, the distal end of one or more of optical fibers 331, 333, 335, 337 are positioned such that light that is emitted from the distal end is biased in a distal direction of catheter 300.

At a proximal region of catheter 300, the enclosed emitters 332, 334, 336, 338 may be optically coupled via optical fibers 331, 333, 335, 337 to one or more light energy sources. In some embodiments, each of the enclosed emitters are coupled to the same light energy source.

For light energy to be absorbed by fluid in the enclosure to generate acoustic energy (e.g., from shock waves), it may be important for enclosed emitters 332, 334, 336, 338 to be spaced from enclosure 310 by a distance equal to or greater than an absorption depth of the light in the fluid. In some embodiments, enclosure 310, during use (e.g., upon inflation), is spaced from enclosed emitters 332, 334, 336, 338 by a distance no less than 1.0 mm. In some embodiments, when the absorption depth of the light is shorter due to properties of one or both of the light or the fluid, the enclosed emitters may be spaced less than 1.0 mm from the enclosure. In some embodiments, the enclosed emitters may be spaced as close as 0.1 mm from the enclosure. The enclosed emitters may be spaced no more than 5.0 mm from the enclosure to ensure sufficient sonic pressure reaches the treatment target.

Elongate shaft 302 includes a guidewire lumen 303 by which the catheter can be delivered to the treatment site. In one or more embodiments, catheter 300 includes additional lumina for delivering fluid (e.g., conductive fluid) to fill or inflate the enclosure. In one or more embodiments, catheter 300 includes additional lumina that extend proximate distal emitters 320 for delivering fluid and/or aspirating the distal treatment region.

As illustrated in FIG. 3B, the enclosed, radially-firing (or side-firing) emitters 332, 334, 336, 338 and optical fibers 331, 333, 335, 337 are embedded in or mounted on an outer shaft 340 that is positioned around elongate member 302. In some embodiments, outer shaft 340 may be formed integrally with elongate member 302. In some embodiments, outer shaft 340 is made of a polymeric material. For example, outer shaft 340 may be made, at least in part, of a polycarbonate, acrylonitrile butadiene styrene, polybutylene terephthalate, polyether ether ketone, a combination of such polymers, or another material having similar properties.

According to one or more embodiments, a dual energy source catheter including a light energy source can selectively launch laser light energy from a laser with a specific wavelength for the most effective ablation of hard and long calcified lesions, as well as removal of soft plaque lesions. In other words, a laser wavelength may be selected based on the type of tissue to be ablated. In some embodiments, with the lesion at least partially opened by laser ablation from unenclosed distal emitters (e.g., distal emitters 220 or 320), the catheter can be further inserted for the electrohydraulic emitters (or another type of radially-firing emitters) to crack calcified lesions.

The wavelengths of lasers for both enclosed and unenclosed types of emitters may be selected to target one or multiple types of lesions. For example, the forward-firing laser wavelength may be selected to optimally target fibrotic tissue for ablation and the radially-firing laser wavelength may be selected to generate acoustic pressure in saline to optimally target calcific lesions. Alternatively, the forward-firing laser wavelength may also be selected to generate acoustic pressures (rather than photoablation) to target lesions of interest. In such embodiments, the catheter may include a photoacoustic transducer for converting light energy to mechanical energy.

In some embodiments, a catheter may include, in an enclosure such as an angioplasty balloon, both electrohydraulic and light-based acoustic energy emitters. Such catheters may include emitter bands and conductive elongate members forming electrode pairs for electrohydraulic emitters and light emitting portions of one or more optical fibers for light-based acoustic energy emitters. Incorporating two types of acoustic energy emitting sources inside the enclosure may allow the catheter to generate acoustic energy with a wider range of acoustic properties to treat, for example, different types of lesions. Additionally, including light-based emitters may help to reduce the crossing profile of catheters that have only electrohydraulic emitters.

In some embodiments, a catheter may include, outside of an enclosure, both electrohydraulic and light-based acoustic energy emitters. Such a catheter may include, at a distal end of the catheter, one or more electrode pairs such as any of the unenclosed electrode pairs described herein, and one or more light emitting regions of one or more optical fibers. In one embodiment, the one or more optical fibers are optically connected to a light energy source that emits light having a wavelength suitable for absorption by a first target tissue. The one or more electrode pairs may be configured to emit acoustic energy tuned to disrupt a second target tissue. In this way, a catheter system having multiple energy sources may be used to treat a lesion having multiple tissue types.

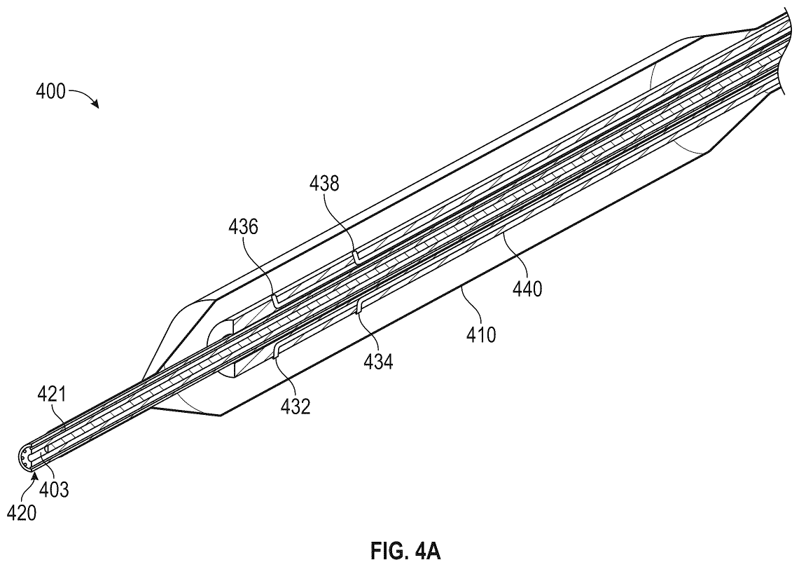

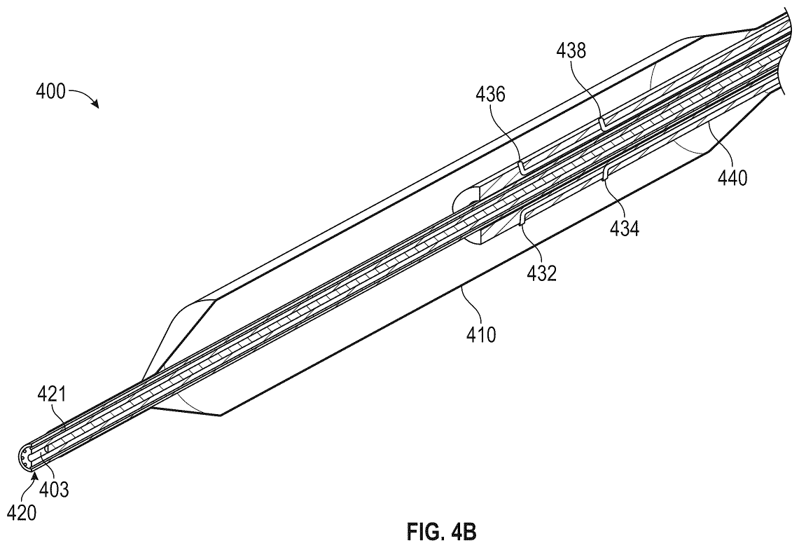

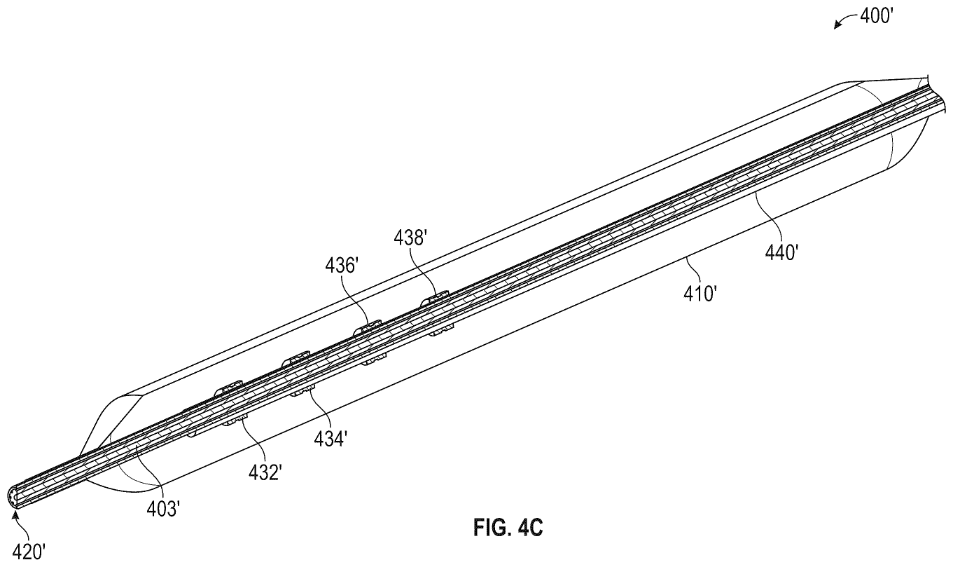

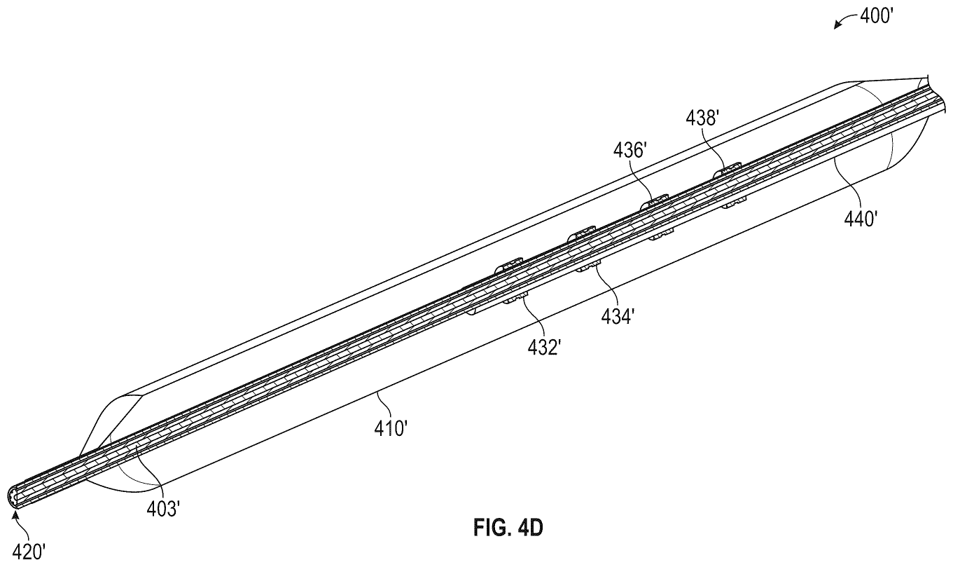

FIGS. 4A and 4B depict cross-sectional views of a distal end of an acoustic energy catheter 400 having longitudinally translatable enclosed acoustic energy emitters, according to one or more embodiments. Catheter 400 includes one or more forward-firing light emitters 420 at its distal end for disrupting an occluded body lumen. Catheter 400 includes enclosed acoustic energy emitters 432, 434, 436, 438 that are longitudinally (in a distal to proximal direction) movable within an enclosure 410 (e.g., an angioplasty balloon) to treat relatively long lesions. Importantly, this movability of acoustic energy emitters 432, 434, 436, 438 allows a long lesion to be treated without having to deflate, move, and reinflate enclosure 410.

Optical fibers 431, 433, 435, 437 may be flexible optical fibers or fiber bundles. Emitters 432, 434, 436, 438 may comprise light emitting regions of optical fibers 431, 433, 435, 437, respectively. For example, a distal end of an optical fiber may form an emitter. In some embodiments, one or more evanescent regions of an optical fiber with thinned cladding may form one or more emitters.

One or more acoustic energy emitters may be assembled on a longitudinally movable outer shaft 440 to target long lesions (e.g., lesions longer than 10 mm). According to these embodiments, balloon dimensions range from 2.5 mm to 12 mm in diameter and longer balloon lengths can be made as long as 200 mm with these movable emitters.

In one or more embodiments, acoustic energy catheter 400 includes a guidewire lumen 403 defining a central axis of catheter 400 and a plurality of optical fibers 421 located around all or part of the guide wire lumen (as shown in FIGS. 4A and 4B). Optical fibers 421 may be optically connected at their proximal ends to the same or a different light energy source as optical fibers 431, 433, 435, 437. In some examples, catheter 400 is configured such that emitters 420 are provided with different laser light energy properties than emitters 432, 434, 436, 438. Emitters 420 may be provided, for example, with light energy having a different wavelength, power density, or pulse width.

FIGS. 4C and 4D depict cross-sectional views of a distal end of an acoustic energy catheter 400′ having longitudinally translatable enclosed acoustic energy emitters, according to one or more embodiments. Catheter 400′ includes one or more forward-firing light emitters 420′ at its distal end for disrupting an occluded body lumen. Catheter 400′ includes enclosed electrohydraulic acoustic energy emitters 432′, 434′, 436′, 438′ that are longitudinally (in a distal to proximal direction) movable within an enclosure 410′ (e.g., an angioplasty balloon) to treat relatively long lesions. Similar to catheter 400, movability of acoustic energy emitters 432′, 434′, 436′, 438′ allows a long lesion to be treated without having to deflate, move, and reinflate enclosure 410′. Acoustic energy catheter 400′ includes a guidewire lumen 403′ defining a central axis of catheter 400 along which acoustic energy emitters 432′, 434′, 436′, 438′ may be movable.

Each acoustic electrohydraulic acoustic energy emitter of catheter 400′ includes one or more electrode pairs that emit shock waves when a high voltage pulse is delivered across a spark gap of the one or more electrode pairs. Electrode pairs may be electrically connected such that a single high voltage pulse from a high voltage generator generates a shock wave from each electrode pair.

One or more acoustic energy emitters may be assembled on a longitudinally movable outer shaft 440′ to target long lesions (e.g., lesions longer than 10 mm). According to these embodiments, balloon dimensions range from 2.5 mm to 12 mm in diameter and longer balloon lengths can be made as long as 200 mm with these movable emitters. Advantageously, electrohydraulic acoustic energy emitters can be electrically connected to a high voltage source by wires that may be easier to incorporate into a movable emitter catheter than light-based acoustic energy emitters, which require coupling of light guides to a light energy source.

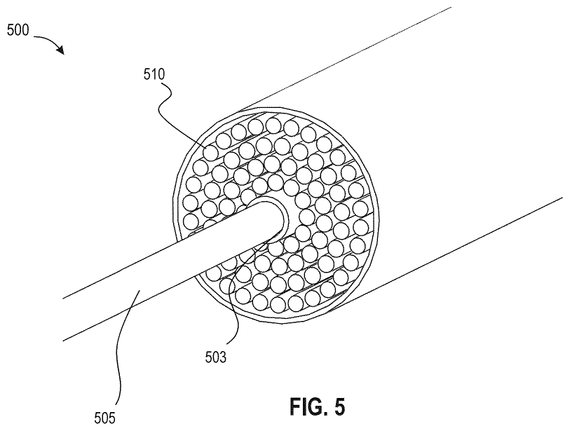

FIG. 5 illustrates a cross-sectional view of a catheter 500 at a proximal location (i.e., proximal of emitters and any enclosure), according to aspects of the disclosure. Catheter 500 includes a guidewire lumen 503 for accommodating a guidewire 505, by which catheter 500 may be delivered to a treatment site. Catheter 500 includes a plurality of optical fibers 510 (e.g., individual fibers or a fiber bundle). At the distal region, these optical fibers may be positioned such that light emitting regions of the optical fibers form the acoustic energy emitters of catheter 500, such as distal unenclosed emitters and enclosed emitters. Guidewire lumen 503 may be centered within the catheter in some embodiments or offset from an axial center in other embodiments. Guide wire lumen may range from 0.01 inch to 0.05 inch. In some examples, the guide wire lumen is 0.014 inch. In some examples, the guide wire lumen is 0.018 inch. In some examples, the guide wire lumen is 0.035 inch. Catheter diameters may range from less than 1 mm to up to 5 mm.

FIG. 8 illustrates an example of a computing system 800 that can be used for one or more components of the system for catheter 100 of FIG. 1, such as subsystem 150 for controlling energy delivery to catheter 100. System 800 can be a computer connected to a network, such as one or more networks of hospital, including a local area network within a room of a medical facility and a network linking different portions of the medical facility, or a wide-area network accessed through the internet or other means. System 800 can be a client or a server. System 800 can be any suitable type of processor-based system, such as a personal computer, workstation, server, handheld computing device (portable electronic device), such as a phone or tablet, or dedicated device. System 800 can include, for example, one or more of input device 820, output device 830, one or more processors 810, storage 840, and communication device 860. Input device 820 and output device 830 can generally correspond to those described above and can either be connectable or integrated with the computer.

Input device 820 can be any suitable device that provides input, such as a touch screen, keyboard or keypad, mouse, gesture recognition component of a virtual/augmented reality system, or voice-recognition device. Output device 830 can be or include any suitable device that provides output, such as a display, touch screen, haptics device, virtual/augmented reality display, or speaker.

Storage 840 can be any suitable device that provides storage, such as an electrical, magnetic, or optical memory including a RAM, cache, hard drive, removable storage disk, or other non-transitory computer-readable medium. Communication device 860 can include any suitable device capable of transmitting and receiving signals over a network, such as a network interface chip or device. The components of the computing system 800 can be connected in any suitable manner, such as via a physical bus or wirelessly.

Processor(s) 810 can be any suitable processor or combination of processors, including any of, or any combination of, a central processing unit (CPU), field programmable gate array (FPGA), and application-specific integrated circuit (ASIC). Software 850, which can be stored in storage 840 and executed by one or more processors 810, can include, for example, the programming that embodies the functionality or portions of the functionality of the present disclosure (e.g., as embodied in the devices as described above), such as programming for performing one or more steps of method 200, method 300, and/or method 600.

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer-readable medium can include, but is not limited to, an electronic, magnetic, optical, electromagnetic, or infrared wired or wireless propagation medium.

System 800 may include a sensor device 870 that provides sensor data for processing by processor 810. Sensor device 870, in some embodiments, may be an imaging sensor that provides imaging data, for a lesion being treated. In some embodiments, sensor device 870 may be a voltage sensor, a current sensor, a pressure sensor, a temperature sensor, an electromagnetic sensor, or an optical sensor for providing data about a state of the catheter or a lesion. Depending on the type of sensors used, sensor device 870 can be physically located on a catheter along a region that is configured to enter a patient or incorporated into a part of the catheter system that remains external to a patient during treatment. In some embodiments, sensor device 870 can include one or more of the sensor types identified here, in any combination. In some implementations, data collected by sensor device 870 may be displayed on output device 830, modified or corrected through input device 820, saved to storage device 840, transmitted to a separate system via communication device 860, or any combination thereof.

System 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, TI or T3 lines, cable networks, DSL, or telephone lines.

System 800 can implement any operating system suitable for operating on the network. Software 850 can be written in any suitable programming language, such as C, C++, Java, or Python. In various examples, application software embodying the functionality of the present disclosure can be deployed in different configurations, such as in a client/server arrangement or through a Web browser as a Web-based application or Web service.

System 800 may be configured to selectively control the delivery of energy from one or more of energy sources (e.g., a voltage pulse generator or a light energy source) to one or more acoustic energy emitters (e.g., a forward-firing emitter, a radially-firing emitter, an unenclosed emitter, or an enclosed emitter) depending on input from input device 820.

System 800 may be configured to tune the energy properties of energy delivered to one or more of the above-described emitters based on tissue properties received from sensor device 870. Tissue properties may include lesion tissue type (e.g., calcific, thrombic, fibrotic), lesion morphology (e.g., thickness, length, eccentricity).

### Methods of Use

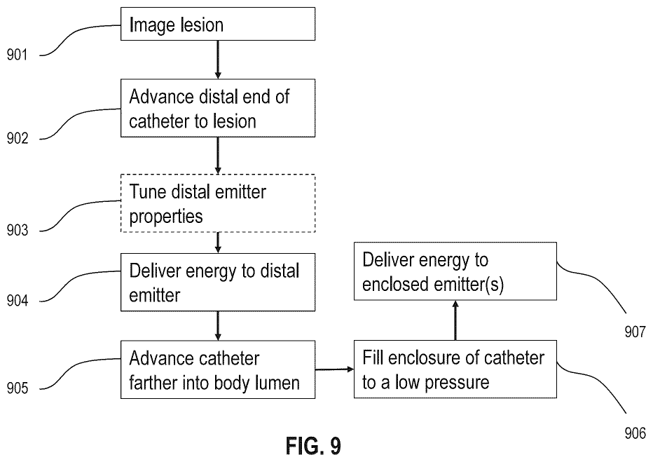

FIG. 9 illustrates an exemplary method of treating stenotic lesions according to one or more aspects of the invention. In one or more embodiments, a method for treating stenotic lesions includes one or more of the steps of: at step 901, imaging the lesion (e.g., by one or more of angiography, intravascular ultrasound, optical coherence tomography); at step 902, advancing a distal end of a dual energy source catheter to the lesion; optionally, at step 903, tuning energy properties of distal emitters (e.g., selecting a laser wavelength or adjusting high voltage pulse electrical properties (amplitude, frequency, pulse width) to ablate the lesion and/or generate cavitation bubbles at distal unenclosed emitters); at step 904, delivering energy to distal emitters (e.g., firing light energy in a forward direction (distally away from a distal end of the catheter) or delivering current across one or more electrode pairs) to generate acoustic energy for disrupting the lesion; at step 905, advancing the catheter farther into the body lumen to position one or more enclosed shock wave emitters adjacent to the lesion; at step 906, filling an enclosure of the catheter with fluid (e.g., inflating to 1 atm-4 atm with saline); and, at step 907, generating shock waves from the one or more enclosed shock wave emitters by delivering energy pulses (e.g., from laser light pulses or high-voltage pulses).

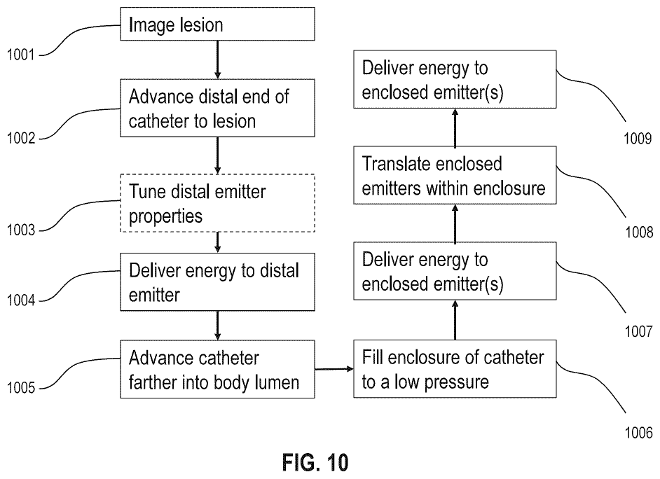

FIG. 10 illustrates an exemplary method of treating stenotic lesions according to one or more aspects of the invention. In one or more embodiments, a method for treating stenotic lesions includes one or more of the steps of: at step 1001, imaging the lesion (e.g., by one or more of angiography, intravascular ultrasound, optical coherence tomography); at step 1002, advancing a distal end of a dual energy source catheter to the lesion; optionally, at step 1003, tuning energy properties of distal emitters (e.g., selecting a laser wavelength or adjusting high voltage pulse electrical properties (amplitude, frequency, pulse width) to ablate the lesion and/or generate cavitation bubbles at distal unenclosed emitters); at step 1004, delivering energy to distal emitters (e.g., firing light energy in a forward direction (distally away from a distal end of the catheter) or delivering current across one or more electrode pairs) to generate acoustic energy for disrupting the lesion; at step 1005, advancing the catheter farther into the body lumen to position one or more radial shock wave emitters adjacent to the lesion; at step 1006, filling an enclosure of the catheter with fluid (e.g., inflating to 1 atm-4 atm with saline); at step 1007, generating shock waves from the one or more enclosed shock wave emitters by delivering energy pulses (e.g., from laser light pulses or high-voltage pulses); at step 1008, translating enclosed shock wave emitters to a different location within the enclosure; and, at step 1009, generating shock waves from the one or more enclosed shock wave emitters by delivering energy pulses.

The elements and features of the example catheters and catheter systems illustrated throughout this specification and drawings may be rearranged, recombined, and modified without departing from the present disclosure. For instance, the number, placement, and spacing of shock wave generating regions or emitters can be modified and the number, placement, and spacing of the enclosures of catheters can be modified without departing from the present disclosure.

Although the catheter devices described herein have been discussed primarily in the context of treating coronary occlusions, such as lesions in vasculature, the catheter devices described herein can be used for a variety of occlusions, such as occlusions in the peripheral vasculature (e.g., above-the-knee, below-the-knee, iliac, carotid, etc.). For further examples, various embodiments may be used for treating soft tissues, such as cancer and tumors (i.e., non-thermal ablation methods), blood clots, fibroids, cysts, organs, scar and fibrotic tissue removal, or other tissue destruction and removal treatments. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal, and destruction of tissue, or used in place of thermal treatments or cauterization for venous insufficiency and fallopian ligation (i.e., for permanent female contraception).

In one or more examples, the electrode assemblies and, catheters described herein could also be used for tissue engineering methods, for instance, for mechanical tissue decellularization to create a bioactive scaffold in which new cells (e.g., exogenous and endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

It will be understood that the foregoing is only illustrative, and that various modifications, alterations and combinations can be made by those skilled in the art without departing from the scope and spirit of the disclosure. Any of the variations of the various catheters disclosed herein can include features described by any other catheters or combination of catheters herein. Furthermore, any of the methods can be used with any of the catheters disclosed. Accordingly, it is not intended that the systems, catheters, and methods described herein be limited, except as by the appended claims.

1. A catheter for treating stenosis in a body lumen, the catheter comprising:
an elongate member;
an enclosure sealed to a distal region of the elongate member and fillable with a fluid;
a forward-firing acoustic energy emitter located on the elongate member at least partially outside of the enclosure on a distal side of the enclosure; and
a radially-firing acoustic energy emitter located on the elongate member at least partially inside of the enclosure.

2. The catheter of claim 1, wherein the forward-firing acoustic energy emitter is optically connected to a light energy source, the light energy source comprises a laser, and the forward-firing emitter comprises a distal end of an optical fiber.

3. The catheter of claim 2, wherein the light energy source emits infrared light.

4. The catheter of claim 1, wherein the radially-firing acoustic energy emitter is electrically connected to a voltage pulse generator and comprises an electrode pair.

5. The catheter of claim 4, wherein a first electrode of the electrode pair comprises a conductive surface of a band and a second electrode of the electrode pair comprises a conductive portion of an elongate conductive member.

6. The catheter of claim 1, wherein the forward-firing acoustic energy emitter is electrically connected to a voltage pulse generator and comprises an electrode pair.

7. The catheter of claim 1, wherein the radially-firing acoustic energy emitter is optically connected to a light energy source that emits an infrared laser light.

8. The catheter of claim 1, comprising a first radially-firing acoustic energy emitter and a second radially-firing acoustic energy emitter.

9. The catheter of claim 8, wherein the first radially-firing acoustic energy emitter is electrically connected to a voltage pulse generator and the second radially-firing acoustic energy emitter is optically connected to a light energy source.

10. The catheter of claim 1, wherein the forward firing acoustic energy emitter and the radially firing acoustic energy emitter are connected to different types of energy sources.

11. The catheter of claim 1, wherein the forward firing acoustic energy emitter and the radially firing acoustic energy emitter are connected to a single energy source.

12. The catheter of claim 11, wherein the forward firing acoustic energy emitter and the radially firing acoustic energy emitter are electrically connected in series.

13. The catheter of claim 11, wherein the forward firing acoustic energy emitter and the radially firing acoustic energy emitter are connected to separate channels of a high-voltage generator.

14. A catheter for treating stenosis in a body lumen, the catheter comprising:
an elongate member;
an enclosure sealed to a distal region of the elongate member;
a longitudinally movable member mounted at least partially around the elongate member and located inside of the enclosure;
a forward-firing acoustic energy emitter located on the elongate member at least partially outside of the enclosure on a distal end of the enclosure; and
a radially-firing acoustic energy emitter located on the longitudinally movable member at least partially inside of the enclosure.

15. The catheter of claim 14, wherein the forward-firing acoustic energy emitter comprises a distal end of an optical fiber that extends from a laser source.

16. The catheter of claim 14, wherein the radially-firing acoustic energy emitter comprises a distal end of an optical fiber that extends from a laser source.

17. The catheter of claim 16, wherein the laser source generates a laser having a wavelength suitable for treating calcified lesions.

18. The catheter of claim 16, wherein the laser source generates a laser having a wavelength suitable for treating tissue softer than calcium.

19. The catheter of claim 14, wherein the enclosure has a working length of at least 30 mm.

20. A method of treating a lesion in a vessel, the method comprising:
advancing a catheter into the vessel until a distal end of the catheter is positioned proximate the lesion;
delivering energy to a distal acoustic energy emitter of the catheter;
advancing the catheter such that an enclosed radially-firing acoustic energy emitter of the catheter is adjacent the lesion;
delivering energy to the enclosed radially-firing acoustic energy emitter to generate acoustic energy to further treat the lesion; and
generating one or more pressure waves from the enclosed radially-firing acoustic energy emitter.

21. The method of claim 20, wherein the enclosed radially-firing acoustic energy emitter comprises one of an electrode pair and a distal end of an optical fiber.

22. The method of claim 20, further comprising:
before the step of delivering energy to the enclosed radially-firing acoustic energy emitter, inflating the enclosure of the catheter;
after the step of delivering energy to the enclosed radially-firing acoustic energy emitter, moving the radially-firing emitter in a longitudinal direction of the catheter;
repeating the step of generating one or more pressure waves from the enclosed radially-firing acoustic energy emitter.

23. The method of claim 20, further comprising the step of tuning acoustic energy properties of the enclosed radially-firing acoustic energy emitter.

24. A catheter system for treating stenosis in a body lumen, the catheter system comprising:
a first energy source;
a second energy source; and
a catheter comprising:
an elongate member configured to be navigated through the body lumen;
a first acoustic energy emitter connected to the first energy source and configured to emit acoustic energy when energy is received from the first energy source; and
a second acoustic energy emitter connected to the second energy source and configured to emit acoustic energy when energy is received from the second energy source.

25. The catheter system of claim 24, wherein the catheter further comprises an enclosure and the first acoustic energy emitter and the second acoustic energy emitter are enclosed in the enclosure.

26. The catheter system of claim 24, wherein the first energy source is a voltage pulse generator and the second energy source is a laser light source.

27. The catheter system of claim 24, wherein the first energy source is a first voltage pulse generator and the second energy source is a second voltage pulse generator configured to generate voltage pulses having different electrical properties than voltage pulses generated by the first voltage pulse generator.

28. The catheter system of claim 24, wherein the first acoustic energy emitter and the second energy emitter are both unenclosed.

29. The catheter system of claim 24, wherein the first energy source is a first laser light source and the second energy source is a second laser light source that generates light having different light energy properties than the light generated by the first laser light source.

30. The catheter system of claim 24, further comprising a third energy source, wherein the catheter includes a third acoustic energy emitter connected to the third energy source.
